(19) United States
(12) Patent Application Publication
(10) Pub. No.: US 2026/0272343 A1
(43) Pub. Date: Sep. 17, 2026

(54) APPARATUS AND METHOD FOR ESTIMATING OXYGEN SATURATION

(71) Applicant: Electronics and Telecommunications Research Institute, Daejeon (KR)

(72) Inventors: Ja Yol LEE, Daejeon (KR); Bon Tae KOO, Daejeon (KR); Youngseok BAEK, Daejeon (KR)

(73) Assignee: Electronics and Telecommunications Research Institute, Daejeon (KR)

(21) Appl. No.: 19/562,935

(22) Filed: Mar. 11, 2026

(30) Foreign Application Priority Data
Mar. 12, 2025 (KR) 10-2025-0031895

(51) Int. Cl. A61B 5/1455; A61B 5/00; A61B 5/021; A61B 5/113

(52) U.S. Cl. CPC A61B 5/14551; A61B 5/02108; A61B 5/113; A61B 5/725

(57) ABSTRACT

A method for measuring a person's oxygen saturation is provided. The method includes: acquiring, by a radar sensor, a radar complex signal reflected from the person; deriving, by a processor, fine chest displacement information of the person based on the radar complex signal; deriving, by the processor, intra-pulmonary pressure information of the person based on the fine chest displacement information; deriving, by the processor, tidal volume information of the person based on the intra-pulmonary pressure information; deriving, by the processor, minute ventilation information of the person based on the tidal volume information; deriving, by the processor, carbon dioxide partial pressure information of the person based on the minute ventilation information; deriving, by the processor, oxygen partial pressure information of the person based on the carbon dioxide partial pressure information; and deriving, by the processor, oxygen saturation information of the person based on the oxygen partial pressure information.

### CROSS-REFERENCE TO RELATED APPLICATIONS

This application claims priority under 35 U.S.C. § 119 to Korean Patent Application No. 10-2025-0031895 filed on Mar. 12, 2025, in the Korean Intellectual Property Office, the disclosure of which is incorporated by reference herein in its entirety.

### BACKGROUND

The present disclosure relates to an apparatus and method for estimating oxygen saturation, and more particularly, to an apparatus and method for estimating the oxygen saturation of a person moving arbitrarily, using a moving average filter and a Butterworth band-pass filter.

As smart devices have advanced, the market for healthcare-related sensing devices has been growing. Accordingly, various devices for measuring oxygen saturation are being developed.

To estimate a person's oxygen saturation as an output value, conventional technology utilizes fine displacement of the upper chest area, respiration rate, heart rate, height, and weight as input values. However, according to conventional technology, unnecessary arbitrary movements of a person cause significant changes in pulmonary ventilation, distorting the estimated tidal volume, which leads to a sharp decrease in the accuracy of oxygen saturation estimation. Consequently, in a post-processing step, an additional identification of the estimated oxygen saturation and the machine learning-based estimation result, which uses the fine displacement of the upper chest area as an input, is performed.

### SUMMARY

An object of the present disclosure is to provide an apparatus and method for estimating the oxygen saturation of a person moving arbitrarily, using a moving average filter and a Butterworth band-pass filter.

According to an embodiment of the present disclosure, a method for measuring a person's oxygen saturation may include: acquiring, by a radar sensor, a radar complex signal reflected from the person; deriving, by a processor, fine chest displacement information of the person based on the radar complex signal; deriving, by the processor, intra-pulmonary pressure information of the person based on the fine chest displacement information; deriving, by the processor, tidal volume information of the person based on the intra-pulmonary pressure information; deriving, by the processor, minute ventilation information of the person based on the tidal volume information; deriving, by the processor, carbon dioxide partial pressure information of the person based on the minute ventilation information; deriving, by the processor, oxygen partial pressure information of the person based on the carbon dioxide partial pressure information; and deriving, by the processor, oxygen saturation information of the person based on the oxygen partial pressure information.

According to an embodiment of the present disclosure, an apparatus for measuring a person's oxygen saturation may include: a radar sensor configured to acquire a radar complex signal reflected from the person; a processor for deriving the person's oxygen saturation information based on the radar complex signal; and a memory for storing command information that directs the operation of the processor. In one example, based on the command information, the processor may derive fine chest displacement information of the person based on the radar complex signal, derive intra-pulmonary pressure information of the person based on the fine chest displacement information, derive tidal volume information of the person based on the intra-pulmonary pressure information, derive minute ventilation information of the person based on the tidal volume information, derive carbon dioxide partial pressure information of the person based on the minute ventilation information, derive oxygen partial pressure information of the person based on the carbon dioxide partial pressure information, and derive the person's oxygen saturation information based on the oxygen partial pressure information.

### BRIEF DESCRIPTION OF THE FIGURES

The above and other objects and features of the present disclosure will become apparent by describing in detail embodiments thereof with reference to the accompanying drawings.

FIG. 1 is a block diagram illustrating an apparatus for estimating oxygen saturation according to an embodiment of the present disclosure.

FIG. 2 is a graph showing the change over time of the phase modulation component of a radar complex signal reflected from a person, according to an embodiment of the present disclosure.

FIG. 3 is a diagram for explaining the fine displacement of a person's chest area from which arbitrary movement has been removed through a moving average filter and a second-order Butterworth band-pass filter, according to an embodiment of the present disclosure.

FIG. 4 is a diagram for explaining the estimated intra-pulmonary pressure according to an embodiment of the present disclosure.

FIG. 5 is a graph showing the change over time of the average tidal volume according to an embodiment of the present disclosure.

FIG. 6 is a flowchart illustrating a method for estimating oxygen saturation according to an embodiment of the present disclosure.

### DETAILED DESCRIPTION

Hereinafter, embodiments of the present disclosure will be described clearly and in detail so that those skilled in the art to which the present disclosure pertains can easily carry out the disclosure.

Terms such as “unit”, “module”, or functional blocks shown in the drawings, as used herein, may be implemented in the form of software configurations, hardware configurations, or combinations thereof. Hereinafter, for the sake of clearly explaining the technical idea of the present invention, detailed descriptions of overlapping components will be omitted.

In the present document, each of the phrases “A or B”, “at least one of A and B”, “at least one of A or B”, “A, B, or C”, “at least one of A, B, and C”, and “at least one of A, B, or C” may include any one of the items listed together in the relevant phrase, or any possible combination of all of them.

The embodiments according to the following descriptions relate to a technology for estimating the oxygen saturation of an arbitrarily moving person using a non-contact radar sensor. More specifically, they relate to a radar signal processing algorithm for more accurately estimating oxygen saturation from the effects of arbitrary motion interference and thermal noise by using a moving average filter and a Butterworth band-pass filter. These will be described in detail with reference to the drawings.

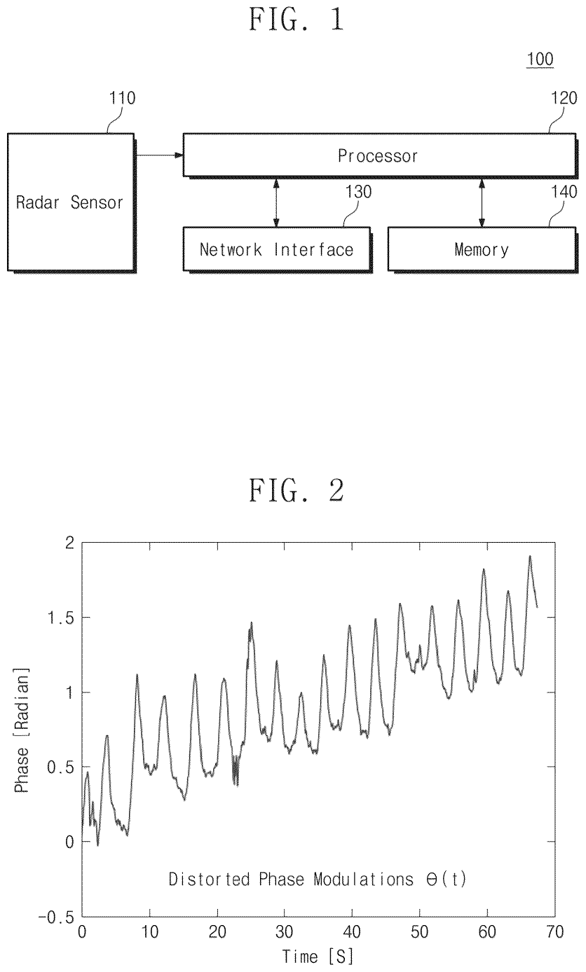

FIG. 1 is a block diagram illustrating an apparatus for estimating oxygen saturation according to an embodiment of the present disclosure. Referring to FIG. 1, the apparatus for estimating oxygen saturation 100 may include a radar sensor 110, a processor 120, a network interface 130, and a memory 140.

The radar sensor 110 of FIG. 1 may be a non-contact radar sensor, for example, a Doppler radar sensor. The apparatus for estimating oxygen saturation 100 may estimate oxygen saturation from a radar complex signal reflected from a person's upper body. Here, data related to one-dimensional fine displacement of the upper chest, respiration rate, and/or heart rate may be extracted from the reflected radar complex signal. The apparatus for estimating oxygen saturation 100 may acquire pulmonary ventilation, tidal volume, and minute ventilation using the one-dimensional fine displacement of the upper chest and respiration rate.

In the present disclosure, the term “fine chest displacement information” may also be expressed as “fine displacement information of the chest,” and the two expressions are used interchangeably to indicate the same information representing fine displacement of a person's chest.

According to an embodiment, carbon dioxide partial pressure PCO₂ and oxygen partial pressure PO₂ may be acquired based on minute ventilation, heart rate, height, and body weight, and oxygen saturation SO₂ may be estimated based on carbon dioxide partial pressure PCO₂ and oxygen partial pressure PO₂.

The processor 120 may perform the function of a central processing unit of the apparatus 100. At least one of the processors 120 may include at least one general-purpose processor such as a Central Processing Unit (CPU), an Application Processor (AP), etc. The processor 120 may also include at least one special-purpose processor such as a Neural Processing Unit, a neuromorphic processor, a Graphics Processing Unit (GPU), etc. The processor 120 may include two or more processors of the same type. In another example, at least one of the processors 120 may be manufactured to implement various Machine Learning or Deep Learning modules.

The network interface 130 provides remote communication with an external device. The network interface 130 may perform wireless or wired communication with the external device. The network interface 130 may communicate with an external device through at least one of various communication types such as Ethernet, Wi-Fi, LTE, 5G mobile communication, etc. For example, the network interface 130 may communicate with an external device of the apparatus 100.

The network interface 130 may receive computational data to be processed by the apparatus 100 from an external device. The network interface 130 may output result data (information) generated (derived) by the apparatus 100 to an external device.

The memory 140 may store command information, data, and process codes that are to be processed (executed) or have been processed (executed) by the processor 120.

The memory 140 may be used as the main memory of the apparatus 100. The memory 140 may include DRAM (Dynamic RAM), SRAM (Static RAM), PRAM (Phase-change RAM), MRAM (Magnetic RAM), FeRAM (Ferroelectric RAM), RRAM (Resistive RAM), etc.

The operations and methods for estimating a person's oxygen saturation by the apparatus 100 and its constituent components according to an embodiment of the present disclosure will be described in detail below with reference to the following drawings. FIG. 2 is a graph showing the change over time of the phase modulation component of a radar complex signal reflected from a person, according to an embodiment of the present disclosure. FIG. 2 shows a distorted phase modulation component due to arbitrary movement.

In one embodiment, the radar complex signal s(t) may be expressed as Equation 1 below.

$$s ( t ) = \sqrt{{P}_{r}} \times exp \{ j ( 4 π / λ ) ( {d}_{c} ( t ) + u ( t ) ) \} + {n}_{θ} ( t ) \qquad [ Equation 1 ]$$

In Equation 1, Pᵣ is the received power, λ is the wavelength of the radar signal, d_{c}(t) is the fine displacement of the upper chest area, u(t) is the movement due to arbitrary motion, and no (t) is the phase component of thermal noise.

θ(t) in Equation 2 below is the phase modulation component of the radar complex signal (s(t)) expressed by applying the arctangent function to the radar complex signal according to Equation 1.

$$θ ( t ) = {tan}^{- 1} ( \frac{imag \{ s ( t ) \}}{real \{ s ( t ) \}} ) \approx \frac{4 π}{λ} ( {d}_{c} ( t ) + u ( t ) ) + {n}_{θ} ( t ) \qquad [ Equation 2 ]$$

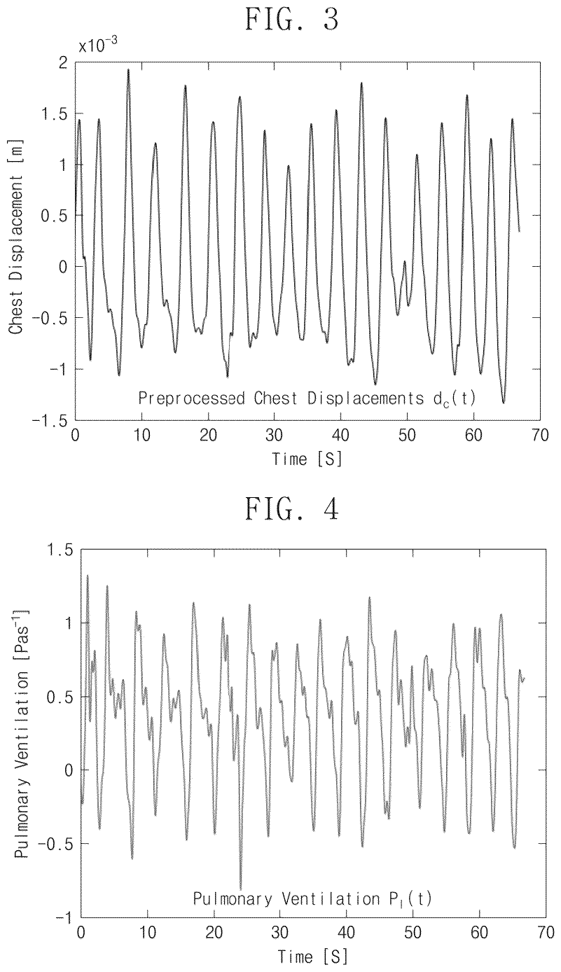

FIG. 3 is a diagram for explaining the fine displacement of a person's chest area from which arbitrary movement has been removed through a moving average filter and a second-order Butterworth band-pass filter, according to an embodiment of the present disclosure.

In one embodiment, when a recursive moving average filter is applied using N_{win} samples of θ(t) from Equation 2, the low-frequency phase modulation component θ_{M}(t) for arbitrary movement may be defined as in Equation 3 below.

$${θ}_{M} ( t ) = {θ}_{M} ( t - 1 ) + \frac{θ ( t ) - ( θ ( t - {N}_{win} )}{{N}_{win}} \qquad [ Equation 3 ]$$

In one embodiment, the phase modulation component due to arbitrary movement may be removed by subtracting the low-frequency phase modulation component θ_{M}(t) from θ(t). For example, the result of subtracting the low-frequency phase modulation component θ_{M}(t) from θ(t), which is denoted as \hat{θ}(t), may be expressed as Equation 4 below.

$$\hat{θ} ( t ) = θ ( t ) - {θ}_{M} ( t ) \approx \frac{4 π}{λ} {d}_{c} ( t ) + {n}_{θ} ( t ) \qquad [ Equation 4 ]$$

In one embodiment, by applying a second-order Butterworth band-pass filter to \hat{θ}(t), the component for residual arbitrary movement and the thermal noise component may be removed. For example, the result of applying a second-order Butterworth band-pass filter with a cutoff frequency Ω_{c} to θ(t) may be expressed as Equation 5 below.

$${Butter}_{{Ω}_{c}} \{ \hat{θ} ( t ) \} \approx \frac{4 π}{λ} {d}_{c} ( t ) \qquad [ Equation 5 ]$$

In one example, the transfer function H_{Butter}{s} of the second-order Butterworth band-pass filter may be configured as in Equation 6 below in the Laplace s domain. In Equation 6, Ω_{c} is the cutoff frequency.

$${H}_{Butter} \{ s \} \approx \frac{{Ω}_{c}^{2}}{{s}^{2} + \sqrt{2} s {Ω}_{c} + {Ω}_{c}^{2}} \qquad [ Equation 6 ]$$

FIG. 4 is a diagram for explaining the estimated intra-pulmonary pressure according to an embodiment of the present disclosure. Specifically, FIG. 4 is a graph showing the change over time of the estimated intra-pulmonary pressure using the respiration rate estimated from fine chest displacement d_{c}(t) and d_{c}(t) from which arbitrary movement has been removed.

In one embodiment, the apparatus and/or method for estimating oxygen saturation calculates the intra-pulmonary pressure Pᵣ (t) using the one-dimensional fine chest displacement d_{c}(t) and the respiration rate f_{RR}, which is the frequency having the maximum value of the one-dimensional fine chest displacement d_{c}(t). For example, d_{c}(t) in Equation 7 is the fine displacement of the person's chest area from which arbitrary movement has been removed, may be derived from Equations 1 to 4 and is based on the measured radar complex signal.

$$\frac{d}{dt} {P}_{I} ( t ) = - g \times BMI \times ( \frac{{d}^{2}}{{dt}^{2}} {d}_{c} ( t ) - {f}_{RR} \times {d}_{c} ( t ) ) \qquad [ Equation 7 ]$$

In Equation 7 above, f_{RR} is the respiration rate corresponding to the frequency that has the maximum value of d_{c}(t), g is the gravitational acceleration, and BMI is the body mass index.

In one embodiment, the apparatus and/or method for estimating oxygen saturation may calculate the tidal volume Vₜ(t) based on the rate of change of the intra-pulmonary pressure P_{I}(t) (e.g., d/dt (P_{I}(t)) derived based on Equation 7), for example, based on Equation 8 below. Here, V_{TLC} is the total tidal volume

$$( e . g . , {V}_{TLC} = 53.5 \times {W}_{t}^{1.06} [ ml ] ) .$$

$${V}_{t} ( t ) = \{ \begin{matrix}{V}_{TLC} \times ( \frac{{P}_{I} ( t - 1 )}{{P}_{I} ( t )} - 1 ) , & {P}_{I} ( t ) \neq 0 \\ 0 , & {P}_{I} ( t ) = 0\end{matrix} \qquad [ Equation 8 ]$$

Equation 9 below illustrates Vₜₕ, which is used to derive the upper boundary value+Vₜₕ and the lower boundary value−Vₜₕ of the change in tidal volume Vₜ(t) in the apparatus and/or method for estimating oxygen saturation. Vₜₕ is the absolute value of the upper and lower boundary values (hereinafter, boundary value).

$${V}_{t h} = 7.69 \times {W}_{t}^{1.04} \qquad [ Equation 9 ]$$

In Equation 9 above, Wₜ is the person's body weight.

In one example, the tidal volume \hat{V}t (t) within the boundary value based on Equation 9 may be derived based on Equation 10 below.

$${\hat{V}}_{t} ( t ) = \{ \begin{matrix}{V}_{t} ( t ) , & \vert {V}_{t} ( t ) \vert \leq {V}_{th} \\ {V}_{t} ( t - 1 ) , & \vert {V}_{t} ( t ) \vert > {V}_{th}\end{matrix} \qquad [ Equation 10 ]$$

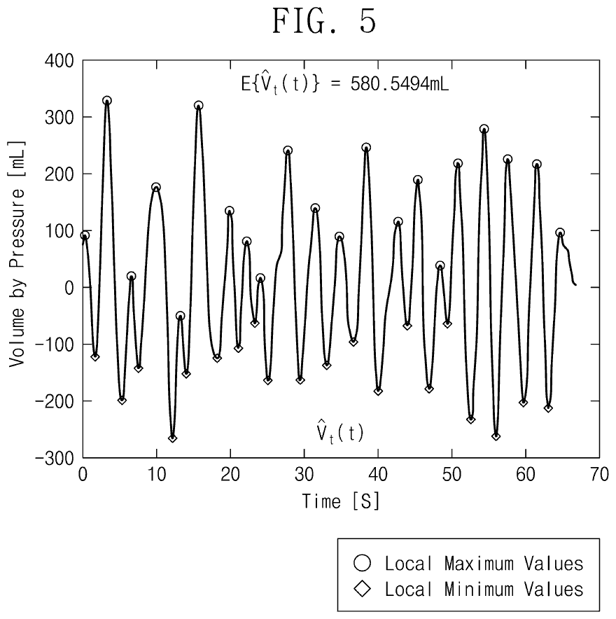

FIG. 5 is a graph showing the change over time of the average tidal volume according to an embodiment of the present disclosure.

Referring to FIG. 5, in an embodiment of the present disclosure, the apparatus and method for estimating oxygen saturation may perform low-pass filtering with the respiration rate f_{RR}, described in conjunction with Equation 7, as the pole for the tidal volume \hat{V}ₜ(t), and then estimate the average tidal volume E{Vₜ(t)} by adding the average of the absolute values of the local maximum and local minimum values. The minute ventilation V_{E}(t) may be derived based on the estimated average tidal volume E{Vₜ(t)}.

For example, the apparatus and method for estimating oxygen saturation may estimate the average tidal volume E{Vₜ(t)} and the minute ventilation V_{E}(t) based on the respiration rate f_{RR} described in conjunction with Equation 7, and for example, may estimate the minute ventilation V_{E}(t) based on Equation 11 below.

$${V}_{E} ( t ) = E \{ {V}_{t} ( t ) \} \times {f}_{RR} \qquad [ Equation 11 ]$$

In an embodiment, the apparatus and method for estimating oxygen saturation may estimate the carbon dioxide partial pressure PCO₂ using the minute ventilation V_{E}(t) (e.g., V_{E}(t) of Equation 11). For example, the apparatus and method for estimating oxygen saturation may estimate the carbon dioxide partial pressure PCO₂ based on Equation 12 below.

$$P {CO}_{2} = \frac{0.041 \times {M}^{1.01} \times {f}_{CR} \times RQ}{( 1 - 1.078 \times {H}^{- 0.289} ) \times {V}_{E} ( t )} \qquad [ Equation 12 ]$$

In Equation 12, Mis body weight, f_{CR} is heart rate, RQ is the respiratory quotient, and His height.

In an embodiment, the apparatus and method for estimating oxygen saturation may estimate the oxygen partial pressure PO₂ using the carbon dioxide partial pressure PCO₂ (e.g., PCO₂ of Equation 12). For example, the apparatus and method for estimating oxygen saturation may estimate the oxygen partial pressure PO₂ based on Equation 13 below.

$${P O}_{2} = {Fi O}_{2} \times ( {P}_{atm} - {P}_{{H}_{2} O} ) - P {CO}_{2} \times ( \frac{1 - {Fi O}_{2} \times ( 1 - RQ )}{RQ} ) \qquad [ Equation 13 ]$$

In Equation 13, FiO₂ is the amount of oxygen in the air (e.g., typically 0.21 on Earth), Pₐₜₘ is the atmospheric pressure (e.g., 760 mmHg at sea level), and P_{H}_{₂}_{O} is the vapor pressure in the alveoli (e.g., typically 47 mmHg).

In an embodiment, the apparatus and method for estimating oxygen saturation may estimate the oxygen saturation SO₂ using the carbon dioxide partial pressure PCO₂ (e.g., PCO₂ of Equation 12) and/or the oxygen partial pressure PO₂ (e.g., PO₂ of Equation 13), and for example, may estimate the oxygen saturation SO₂ based on Equation 14 below.

$${S O}_{2} = 1 - {f}_{A} ( t ) \times {f}_{P {O}_{2}} ( t ) \times exp \{ - {f}_{{PCO}_{2}} ( t ) \times {P O}_{2} \} \qquad [ Equation 14 ]$$

In one example, f_{A}(t) of Equation 14 above may be derived based on Equation 15 below.

$${f}_{A} ( t ) = \{ \begin{matrix}{( β ( t ) - α ( t ) )}^{- 1} , & ζ ( t ) > 1 \\ 1 , & ζ ( t ) = 1 \\ \frac{{ω}_{n} ( t )}{{ω}_{sin} ( t )} , & 0 < ζ ( t ) < 1\end{matrix} \qquad [ Equation 15 ]$$

In one example, f_{PCO}_{₂}(t) of Equation 14 above may be derived based on Equation 16 below.

$${f}_{{PCO}_{2}} ( t ) = \{ \begin{matrix}0 , & ζ ( t ) > 1 \\ {ω}_{n} ( t ) , & ζ ( t ) = 1 \\ γ ( t ) , & 0 < ζ ( t ) < 1\end{matrix} \qquad [ Equation 16 ]$$

In one example, f_{PO}_{₂}(t) of Equation 14 above may be derived based on Equation 17 below. For example, PO₂ of Equation 17 may be based on Equation 13.

$${f}_{P {O}_{2}} ( t ) = \{ \begin{matrix}{f}_{αβ} ( t ) & ζ ( t ) > 1 \\ 1 + {ω}_{n} ( t ) \times {P O}_{2} , & ζ ( t ) = 1 \\ {f}_{sin} ( t ) , & 0 < ζ ( t ) < 1\end{matrix} \qquad [ Equation 17 ]$$

In one example, ζ(t) used in Equations 15 to 17 may be derived based on Equation 18 below, and PCO₂ of Equation 18 may be derived based on Equation 12 above.

$$ζ ( t ) = 1.31 \times {\{ {P CO}_{2} \}}^{- 0.45} + 0.73 \qquad [ Equation 18 ]$$

In one example, ωₙ(t) used in Equations 15 to 17 may be derived based on Equation 19 below, and PCO₂ of Equation 19 may be derived based on Equation 12 above.

$${ω}_{n} ( t ) = 0.28 \times {\{ {P C O}_{2} \}}^{- 0.45} \qquad [ Equation 19 ]$$

In one example, ωₛᵢₙ(t) used in Equation 15 may be derived based on Equation 20 below, PCO₂ of Equation 20 may be derived based on Equation 12 above, ωₙ(t) may be based on Equation 19, and/or ζ(t) may be based on Equation 18.

$${ω}_{sin} ( t ) = {ω}_{n} ( t ) \times \sqrt{1 - ζ ( t )} \qquad [ Equation 20 ]$$

In one example, α(t) used in Equation 15 may be derived based on Equation 21 below, ωₙ(t) of Equation 21 may be based on Equation 19, and/or ζ(t) may be based on Equation 18.

$$α ( t ) = {ω}_{n} ( t ) \times \{ - ζ ( t ) + \sqrt{{ζ}^{2} ( t ) - 1} \} \qquad [ Equation 21 ]$$

In one example, γ(t) used in Equation 15 may be derived based on Equation 22 below, ωₙ(t) of Equation 22 may be based on Equation 19, and/or ζ(t) may be based on Equation 18.

$$β ( t ) = {ω}_{n} ( t ) \times \{ - ζ ( t ) + \sqrt{{ζ}^{2} ( t ) - 1} \} \qquad [ Equation 22 ]$$

In one example, γ(t) used in Equation 16 may be derived based on Equation 23 below, ωₙ(t) of Equation 23 may be based on Equation 19, and/or ζ(t) of Equation 23 may be based on Equation 18.

$$γ ( t ) = {ω}_{n} ( t ) \times ζ ( t ) \qquad [ Equation 23 ]$$

In one example, fₛᵢₙ(t) used in Equation 17 may be based on Equation 24, ωₛᵢₙ(t) of Equation 24 may be based on Equation 20, PO₂ may be based on Equation 13, and/or ζ(t) may be based on Equation 18.

$${f}_{s i n} ( T ) = sin \{ {ω}_{s i n} ( t ) \times {P O}_{2} + {cos}^{- 1} ( ζ ( t ) ) \} \qquad [ Equation 24 ]$$

In one example, f_{αβ}(t) used in Equation 17 may be based on Equation 25, a (t) of Equation 24 may be based on Equation 21, (t) may be based on Equation 22, and PO₂ may be based on Equation 13.

$${f}_{αβ} ( t ) = β ( t ) exp \{ α ( t ) \times {P O}_{2} \} - α ( t ) exp \{ β ( t ) \times {P O}_{2} \} \qquad [ Equation 25 ]$$

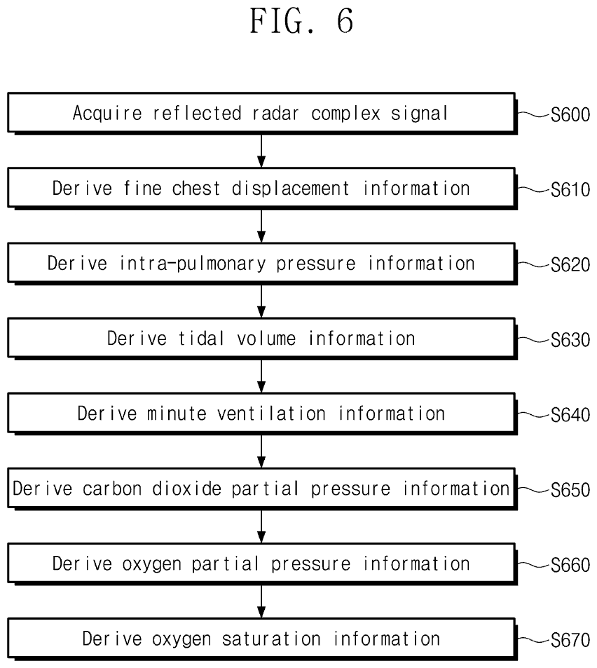

FIG. 6 is a flowchart illustrating a method for estimating oxygen saturation according to an embodiment of the present disclosure. The method according to FIG. 6 will be described together with FIGS. 1 to 5 and the equations described above. The method according to FIG. 6 may include steps S600 to S670 and may be performed by the apparatus 100 of FIG. 1.

In S600, the radar sensor 110 may acquire a radar complex signal (e.g., s(t) of Equation 1) reflected from a person.

In S610, the processor 120 may derive fine chest displacement information of the person based on the radar complex signal acquired in step S600. In one example, the processor 120 may remove the component due to the person's arbitrary movement from the radar complex signal by applying a moving average filter and a Butterworth filter.

In one embodiment, the processor 120 derives the phase modulation component (e.g., θ(t) of Equation 2) of the radar complex signal, derives the low-frequency phase modulation component (e.g., θ_{M}(t) of Equation 3) due to the person's arbitrary movement based on the phase modulation component of the radar complex signal, and may derive the difference (e.g., \hat{θ}(t) of Equation 4) between the phase modulation component and the low-frequency phase modulation component. For example, the processor 120, based on command information, may perform filtering by applying a moving average filter to derive the low-frequency phase modulation component due to the person's arbitrary movement based on the phase modulation component of the radar complex signal. The fine chest displacement information (e.g., d_{c}(t)) of the person may be derived based on the result (e.g., the result of Equation 5) of filtering by applying (by the processor 120) a Butterworth filter to the difference between the phase modulation component and the low-frequency phase modulation component.

In S620, the processor 120 may derive the intra-pulmonary pressure information (e.g., P_{I}(t) of Equation 7) of the person based on the fine chest displacement information. In one example, the processor 120 may derive the rate of change information of the person's intra-pulmonary pressure (e.g., d/dt (P_{I}(t)) of Equation 7) based on the fine chest displacement information.

In S630, the processor 120 may derive the tidal volume information of the person based on the intra-pulmonary pressure information. In one example, the processor 120 may derive the tidal volume information (e.g., Vₜ(t) of Equation 8) of the person based on the rate of change information of the intra-pulmonary pressure (e.g., d/dt (P_{I}(t)) of Equation 7).

In S640, the processor 120 may derive the minute ventilation information of the person based on the tidal volume information. In one example, the processor 120, based on command information, in order to derive the minute ventilation information of the person based on the tidal volume information, may derive the boundary value of the change in tidal volume (e.g., Vₜₕ of Equation 9), derive the average tidal volume information of the person (e.g., the aforementioned E{Vₜ(t)}) based on the boundary value and the maximum frequency value that the fine chest displacement information has (e.g., the aforementioned f_{RR}), and derive the minute ventilation information (e.g., V_{E}(t) of Equation 11) based on the average tidal volume. For example, the boundary value may be derived based on the person's body weight (the aforementioned Wₜ).

In S650, the processor 120 may derive the carbon dioxide partial pressure information of the person based on the minute ventilation information. The derivation of the carbon dioxide partial pressure information according to step S650 may be based on the estimation of carbon dioxide partial pressure according to Equation 12 described above.

In S660, the processor 120 may derive the oxygen partial pressure information of the person based on the carbon dioxide partial pressure information. The derivation of the oxygen partial pressure information according to step S660 may be based on the estimation of oxygen partial pressure according to Equation 13 described above.

In S670, the processor 120 may derive the oxygen saturation information of the person based on the oxygen partial pressure information. The derivation of the oxygen saturation information according to step S670 may be based on the estimation of oxygen saturation according to Equations 14 to 25 described above.

The estimation of oxygen saturation based on the equations of the present disclosure may be based on an algorithm performed by the processor 120 based on command information stored in the memory 140.

In the embodiments according to the descriptions above, an apparatus and method for acquiring oxygen saturation using a non-contact Doppler radar sensor on an arbitrarily moving person may be provided, and an apparatus and method for extracting fine displacement of the upper chest area by compensating for the distorted phase component due to arbitrary movement through a moving average filter may be provided. Furthermore, an apparatus and method for extracting fine displacement of the upper chest area by compensating for the effects of residual arbitrary movement and thermal noise through a second-order Butterworth band-pass filter may be provided.

According to the present disclosure, by utilizing a moving average filter and a second-order Butterworth band-pass filter, the influence of arbitrary movements may be reduced, and the accuracy of oxygen saturation estimation may be improved through a non-complex signal processing procedure.

In the embodiments described above, components according to the technical idea of the present invention were described using terms such as first, second, third, etc. However, terms like first, second, third, etc., are used to distinguish the components from each other and do not limit the present invention. For example, terms like first, second, third, etc., do not imply order or any form of numerical meaning.

The content described above are specific examples for carrying out the present invention. The present invention will not only include the embodiments described above but also embodiments that can be simply designed or easily modified. Furthermore, the present invention will also include technologies that can be easily modified and implemented in the future using the embodiments described above.

What is claimed is:

1. A method for measuring a person's oxygen saturation, the method comprising:
acquiring, by a radar sensor, a radar complex signal reflected from the person;
deriving, by a processor, fine chest displacement information of the person based on the radar complex signal;
deriving, by the processor, intra-pulmonary pressure information of the person based on the fine chest displacement information;
deriving, by the processor, tidal volume information of the person based on the intra-pulmonary pressure information;
deriving, by the processor, minute ventilation information of the person based on the tidal volume information;
deriving, by the processor, carbon dioxide partial pressure information of the person based on the minute ventilation information;
deriving, by the processor, oxygen partial pressure information of the person based on the carbon dioxide partial pressure information; and
deriving, by the processor, oxygen saturation information of the person based on the oxygen partial pressure information,
wherein deriving the fine chest displacement information of the person based on the radar complex signal includes removing a component due to the person's arbitrary movement from the radar complex signal by applying a moving average filter and a Butterworth filter.

2. The method of claim 1, wherein deriving the fine chest displacement information of the person based on the radar complex signal comprises:
deriving a phase modulation component of the radar complex signal;
deriving a low-frequency phase modulation component due to the person's arbitrary movement based on the phase modulation component of the radar complex signal; and
deriving a difference between the phase modulation component and the low-frequency phase modulation component.

3. The method of claim 2, wherein deriving the low-frequency phase modulation component due to the person's arbitrary movement based on the phase modulation component of the radar complex signal is based on filtering by application of the moving average filter.

4. The method of claim 2, wherein the fine chest displacement information of the person is derived based on a result of filtering by application of the Butterworth filter to the difference between the phase modulation component and the low-frequency phase modulation component.

5. The method of claim 1, wherein deriving the minute ventilation information of the person based on the tidal volume information comprises:
deriving a boundary value of a change in the person's tidal volume;
deriving average tidal volume information of the person based on the boundary value and a maximum frequency value that the fine chest displacement information has; and
deriving the minute ventilation information of the person based on the average tidal volume,
wherein the boundary value is derived based on the person's body weight.

6. An apparatus for measuring a person's oxygen saturation, comprising:
a radar sensor configured to acquire a radar complex signal reflected from the person;
a processor configured to derive the person's oxygen saturation information based on the radar complex signal; and
a memory configured to store command information that directs the operation of the processor,
wherein, based on the command information, the processor:
derives fine chest displacement information of the person based on the radar complex signal,
derives intra-pulmonary pressure information of the person based on the fine chest displacement information,
derives tidal volume information of the person based on the intra-pulmonary pressure information,
derives minute ventilation information of the person based on the tidal volume information,
derives carbon dioxide partial pressure information of the person based on the minute ventilation information,
derives oxygen partial pressure information of the person based on the carbon dioxide partial pressure information, and
derives the person's oxygen saturation information based on the oxygen partial pressure information,
wherein the processor removes a component due to the person's arbitrary movement from the radar complex signal by applying a moving average filter and a Butterworth filter, to derive the fine chest displacement information of the person based on the radar complex signal.

7. The apparatus of claim 6, wherein the processor, based on the command information, to derive the fine chest displacement information of the person based on the radar complex signal:
derives a phase modulation component of the radar complex signal,
derives a low-frequency phase modulation component due to the person's arbitrary movement based on the phase modulation component of the radar complex signal, and
derives a difference between the phase modulation component and the low-frequency phase modulation component.

8. The apparatus of claim 7, wherein the processor performs filtering by application of the moving average filter, to derive the low-frequency phase modulation component due to the person's arbitrary movement based on the phase modulation component of the radar complex signal.

9. The apparatus of claim 7, wherein the fine chest displacement information of the person is derived based on a result of filtering by application of the Butterworth filter to the difference between the phase modulation component and the low-frequency phase modulation component.

10. The apparatus of claim 6, wherein the processor, based on the command information, to derive the minute ventilation information of the person based on the tidal volume information:
derives a boundary value of a change in the person's tidal volume,
derives average tidal volume information of the person based on the boundary value and a maximum frequency value that the fine chest displacement information has, and
derives the minute ventilation information based on the average tidal volume,
wherein the boundary value is derived based on the person's body weight.
